(19) United States
(12) Patent Application Publication
(10) Pub. No.: US 2006/0088575 A1
(43) Pub. Date: Apr. 27, 2006

(54) Homeopathic preparations of purified growth factors and purified growth hormones and associated carriers

(76) Inventor: Barbara A. Brewitt, Seattle, WA (US)

Correspondence Address: Victor N. King;SPECKMAN LAW GROUP PLLC, 1501 Western Avenue, Suite 100, Seattle, WA, 98101, US

(21) Appl. No.: 11/242,988

(22) Filed: Oct. 4, 2005

Related U.S. Application Data
(63) Continuation-in-part of application No. 10/001,367, filed on Oct. 30, 2001.
Application No. 10/001,367 is a continuation-in-part of application No. 09/870,132, filed on May 29, 2001, now abandoned.
Application No. 09/870,132 is a continuation of application No. 09/251,820, filed on Feb. 17, 1999, now Pat. No. 6,239,105.
Application No. 09/251,820 is a continuation-in-part of application No. 08/855,096, filed on May 13, 1997, now Pat. No. 6,024,734.
Application No. 08/855,096 is a continuation-in-part of application No. 08/710,040, filed on Sep. 10, 1996, now Pat. No. 5,629,286.
Application No. 08/710,040 is a continuation of application No. 08/488,722, filed on Jun. 8, 1995, now abandoned.
Application No. 08/488,722 is a continuation-in-part of application No. 08/221,365, filed on Mar. 31, 1994, now abandoned.

(51) Int. Cl. A61K 38/18; A61K 33/00; A61K 47/00

(52) U.S. Cl. 424/439; 514/12; 424/600

(57) ABSTRACT

A preparation comprising a homeopathic potency of a purified composition, such as insulin-like growth factor, insulin-like growth factor-1 (IGF-1), growth hormone, transforming growth factor (TGF), TGF_{β}1, fibroblast growth factor (FGF), FGF1, FGF2, GM-CSF, M-CSF, PDGF_{ββ}, nerve growth factor (NGF) and epidermal growth factor (EGF), is provided. The concentration of the purified composition is less than 1×10⁶ molar and the homeopathic potency is formulated in a generally solid, food-based or a generally liquid beverage carrier.

### CROSS-REFERENCE TO RELATED APPLICATIONS

This application is a continuation-in-part of U.S. patent application Ser. No. 10/001,367, filed Oct. 30, 2001, which is a continuation-in-part of U.S. patent application Ser. No. 09/870,132, filed May 29, 2001, now abandoned, which is a continuation of U.S. patent application Ser. No. 09/251,820, filed Feb. 17, 1999, issued May 29, 2001 as U.S. Pat. No. 6,239,105, which is a continuation-in-part of U.S. patent application Ser. No. 08/855,096, filed May 13, 1997, issued Feb. 15, 2000 as U.S. Pat. No. 6,024,734, which is a continuation-in-part of U.S. patent application Ser. No. 08/710,040, filed Sep. 10, 1996, issued May 13, 1997 as U.S. Pat. No. 5,629,286, which is a continuation of U.S. patent application Ser. No. 08/488,722, filed Jun. 8, 1995, now abandoned, which is a continuation-in-part of U.S. patent application Ser. No. 08/221,365, filed Mar. 31, 1994, now abandoned.

### FIELD OF THE INVENTION

The present invention relates to homeopathic preparations of purified proteins, such as growth factors, growth hormone and related molecules, cyclins, and other proteins and peptides, as well as systems and methods for delivery of such preparations and treatment of disorders and conditions by administering such preparations.

### BACKGROUND OF THE INVENTION

Hormones and polypeptide growth factors are important regulatory substances that are involved in the regulation of cell growth and differentiation, as well as in the control of specific metabolic processes. Hormones are defined as chemical messengers that are synthesized in the endocrine glands and secreted into extracellular body fluids. Hormones are transported to hormone-responsive cells, where they bind to a hormone receptor, and the hormone-receptor complex regulates and modulates differentiated functions. Polypeptide growth factors are produced and secreted by cells from a variety of tissues, and are generally involved in paracrine and autocrine responses after activating specific cell receptors. Growth factors are involved in cell survival and play a crucial role in the control mechanisms governing the development and maintenance of organs and tissues. In addition to their growth promoting and differentiation inducing effects, growth factors are also involved in important physiological processes such as inflammation, immune reactions, and tissue and DNA repair.

Specific hemopoietic growth factors have been used to treat diseases such as AIDS and cancer. Hemopoietic growth factors are logical therapeutic immunomodulators to use for treatment of chronic viral infections and other diseases for several reasons. Endogenous growth factors such as granulocyte-macrophage colony stimulating factor (GM-CSF) and macrophage colony stimulating factor (M-CSF) stimulate proliferation of hemopoietic progenitor cells. Lymphocytes, macrophages and natural killer cells that normally produce these factors are quantitatively and qualitatively defective after infection by HIV, HH6V, EBV and other viruses and bacteria. Primates infused with GM-CSF showed low toxicity with some positive but inconsistent rises in platelet number.

Clinical studies on AIDS patients using the growth factors GM-CSF and M-CSF at pharmacological concentrations (ug/kg/day) have produced mixed results. For example, injections or intravenous administration of GM-CSF at concentrations of 0.5-0.8 ug/kg/day transiently increased leukocyte, neutrophil, eosinophil and monocyte counts in AIDS patients with no significant rise in platelet counts or change in reticulocyte and lymphocyte counts (Miles, S. 1992 AIDS Res. Hum. Retroviruses 8:1073-1080). Subcutaneous injections of 0.25-4.0 ug/kg/day improved leukocyte counts with no improvement in hemoglobin or platelet counts. However, the side effects included increased HIV replication, increased levels of P24 antigen, chills, nausea, myalgia and flu-like symptoms (Poli, G. et al. 1991 J. Exp. Med. 173:589-597; Scadden, D. T. 1990 Hematopoietic Growth Factors in Trans. Med., Wiley-Liss Inc., New York, pp. 163-176). GM-CSF also occasionally caused thrombocytopenia. Granulocyte colony stimulating factor (G-CSF) has been effective in correcting neutropenia with some minor increases in lymphocyte counts. Additionally, hemoglobin and reticulocytes increased in numbers in patients given G-CSF alone or in combination with erythropoietin. However, resumption of treatment with AZT after use of these growth factors led to severe anemia. Pharmacological doses of growth factors often have harsh side effects.

Following puberty, there is an exponential decline in growth hormone (Rudman, D, 1985, J. A. Ger. Soc., 33:800-807). By thirty years of age, the normal physiological concentration found in the blood stream is 20 ng/ml (Corpas, E., Harman, S., Pineyro, M., Robertson, R., Blackman, M., 1992, J. Clin. Endocrinol. Metab., 75:530-535). This is reduced to 10 ng/ml by age 60, and continues to decline 2-4 ng/ml each decade (Irranmanesh, A., Lisarraide, G., Veldhuis, J., 1991, J. Clin. Endocrinol. Metab., 73:1081-1088). Additional studies have shown that growth hormone secretion peaks at approximately 31 years of age and then continues to decline by 14 to 50% per decade, dependent on gender, activity level and diet, or with the onset of chronic disease (Ho, K., Veldhuis, J., Endocrinol. Metab., 1994 1 (Suppl A):61-63). While the definition of GH deficiency is not absolute, symptoms associated with age-related declines in hGH are often used to define GH deficiency. The American Association of Clinical Endocrinology and the American College of Endocrinology suggest that growth hormone deficiency is characteristically defined as a cluster of self perceived symptoms which include fatigue, decreased lean body mass, decreased muscle mass, abdominal obesity, reduced cardiac performance, poor sense of well being, poor sleep, decreased physical strength, cold extremities and reduction in skin thickness.

Growth hormone has been isolated and purified from mammalian sources and has been produced recombinantly. Administration of pharmacological dosages of growth hormone is best known for the treatment of growth hormone deficiency disorder in children. Other pharmaceutical indications for growth hormone include: reducing blood pressure and improving cardiovascular function; increasing serum IGF-1 levels; treating growth deficiency disorders; increasing lean body mass, muscle mass and physical strength; improving pulmonary function, vascular and intracellular nutrient support; revitalizing liver, spleen, and brain functions; increasing libido and sex hormones; improving lipoprotein balance and fatty acid levels; increasing energy levels, oxygen uptake, nitrogen retention, physical mobility and exercise performance; eliminating cellulite and improving cholesterol profile; promoting hair growth; improving dermal cellularity, thickness and collagenicity; increasing cartilage strength; increasing the size and function of the thymus and spleen; enhancing immune system function and lymphocyte count; and reducing body fat. Pharmacological application of growth hormone has been shown to improve short term memory; reduce the sense of social isolation; improve REM sleep quality, improve vision, remove wrinkles, quicken wound healing, and generally contribute to a feeling of well-being. Additionally, homeopathic preparations of the present invention may be used to treat AIDS wasting syndrome, autism, Turner syndrome, osteoporosis, Parkinson's, Alzheimer's disease, Down's syndrome and skin resiliency.

Administration of higher than physiological concentrations of growth hormone does, however, produce serious side effects, including increased tissue turgor, neuropathy, back pain, increase in liver enzymes aspartate aminotransferase (SGOT) and alanine aminotransferase SPGT, increased sweating, headache, skin and joint problems, hypertension, edema, cardio-vascular and heart disease, loss of lean mass, carpal tunnel syndrome, musculoskeletal distress, allergic reactions, acute pancreatitis, nausea, insulin resistance, glucose intolerance, soium retension, intracranial hypertension in short stature children, vomiting, pain, arthralgia, paraesthesia, rhinitis, myalgia, flu-like symptoms, leukemia, diabetes, diabetic angiopathy, anemia, excessive rise in IGF-1, fever, suppression of TSH levels, albuminia, gonadal insufficiency, retinopathy, anorexia, hyperglycemia, kidney mass increase and upper respiratory tract infections. It would thus be desirable to identify compositions or means of administration that, when administered, produce the benefits of growth hormone without producing the serious side effects.

Homeopathy, which dates back to the nineteenth century, is founded on the principles of pharmacology and biology. In 1877, Hugo Schultz postulated that the effect of a stimulus on a living cell is indirect and proportional to its intensity and quantity. Later, in 1888, Schultz demonstrated that very low concentrations of yeast toxins increased yeast growth over 100 fold. Concurrently, the psychiatrist Rudolph Arndt developed his “Basic Law of Biology,” which states that weak stimuli slightly accelerate the vital activity, middle-strong stimuli raise it, strong stimuli suppresses it, and very strong stimuli halt vital activity. These separate observations were formulated by Arndt in 1888 into one of the earliest laws of pharmacology representing the homeopathic effect, the Arndt-Schultz law, which states: every stimulus on a living cell elicits an activity, which is inversely proportional to the intensity of the stimulus (Martius F. Das Arndt-Schultz Grundgesetz, Muench Med. Wschr., 1923, 70(31):1005-1006). This law was later restated by Hueppe as: for every substance, small doses stimulate, moderate doses inhibit, large doses kill. Allopathic medicine, with its emphasis on moderate drug doses, works to inhibit undesired physical symptoms and to kill undesired pathogens. Homeopathic medicine begins with small doses and moves towards higher and higher dilutions to stimulate the body's own natural electromagnetic forces.

A common principle of homeopathy is the Law of Similars, which was founded in the science of pharmacology and states that a drug has two effects on the body, a direct effect and the subsequent reaction of the body to the drug, evoking symptoms or side effects. In homeopathy, as the drug is diluted, some of the positive benefits of the drug remain, plus new characteristics of the drug become available to the body which not only alleviate side effects, but have new characteristic features that actually ameliorate other symptoms the person may have.

Homeopathic and allopathic principles can be represented on the same sinusoidal curve, as illustrated in prior related patents incorporated herein by reference. There are several harmonic concentrations over a log scale of dilutions that give the same desired effect. Oscillatory data demonstrating the stimulating and inhibiting effect of log dilutions of anti-IgE antisera which caused human basophil degranulation have been generated and reproduced (Davenas, E., Beauvais, F. et al. Nature 333:816-818, 1988; Beneviste, J., Davenas, E. et al. C.R. Acad. Sci. Paris 312, series II, pp. 461-466, 1991). Control studies using dilutions of antihuman IgG antisera or simply distilled water did not produce this same effect.

This phenomena of oscillatory or polyphasic activity is further described by Bellavite, P. and Signorini, A., in Homeopathy: A Frontier in Medical Science, North Atlantic Press, Berkeley Calif., 1995 at page 130. The essential constituents of homeostatic biological systems susceptible to polyphasic activity based on concentration of drug or growth factor have the following characteristics. First, there are adjustable and reversible effector functions, such as occurs in the endocrine gland. Second there are signal molecules that enable nearby and remote structures to communicate via feedback systems, such as neurotransmitters, hormones, local chemical mediators and cytokines/growth factors. A particular feature of the signaling molecules is that their message is never wholly specific; the same molecules can be used to communicate between different physiological systems. There is a substantial degree of redundancy of biological information, which enables complex biological systems a considerable measure of flexibility. This inherent complexity makes it difficult to develop a rigid schematization of the events following the production of a certain mediator in given pathophysiological conditions. Third, there are cell surface receptors involved, which are capable of increasing, by hypersensing or priming, or decreasing, by desensitization, tolerance, adaptation or downregulation, the number of receptors according to their needs.

Additionally, the cell surface receptors are capable of regulating activity by modifying the affinity for the signal molecules. On occasion, the cells present more than one receptor for the same molecule, but with different affinities and intracellular effects. Lastly, that the multiform characteristics of the signal transduction systems have a level of responsiveness that is also controlled by such systems in the cell, and that they are also modified in the course of disease and are highly susceptible to concentration-specific modulation.

One of the basic tenets of homeopathic medicine is that a cure for a disease can be evoked by using a high dilution medicine that resembles but is different from the cause of the disease. Homeopathy is widely accepted as a useful therapeutic throughout Europe, the British Commonwealth countries and India, and has been demonstrated to have characteristic and reproducible effects. A critical review of more than 100 controlled and/or clinical studies of homeopathy determined that patients received positive healing benefits from homeopathy beyond the placebo effect (Kleijnen, J. et al. 1991 Brit. Med. J. 302:316-323; Linde, K., Clausius, N., Ramirez, G., Melchart, D., Eitel, F., Hedges, L. V., Jonas, W. B., 1997, Lancet, 350:834-843; Reilly, D., et al, 1994, Lancet, 344:1601-1608).

Many homeopathic medicines are used at concentrations of micrograms (10⁻⁶ M) and nanograms (10⁻¹² M); however, in other homeopathic preparations, the dilutions exceed Avogadro's number (6.023×10⁻²³). When homeopathic compounds are diluted 1:10, with repeated succusions (similar to vortexing) and repetitively diluted by this procedure at least 24 times, a potency is achieved that is so highly dilute that the probability of a single molecule of the original substance remaining in the volume used is less than 1×10⁻¹0. Homeopathic practitioners believe that the potency of a compound increases with increasing dilutions. In traditional homeopathic practice, the standard homeopathic dosage is 10-15 drops of a 6 C solution administered two to three times per day. A 30 C homeopathic preparation may be given one to three times per day. A 200 C homeopathic preparation may be given only one time per month or year. A 6 C homeopathic potency approximates ng/ml molar concentrations, which are used in cell culture but would be considered a lower than physiological dose when administered to a patient either orally, topically or by injection.

Highly dilute homeopathic medicines have been effective in treating some conditions, including viral infections, in vivo. Homeopathic potencies of 100 C to 1M of typhoidinum, hydrophobinum, tuberculinum, nux vomica and malandrinum 100% inhibited pock-like lesions caused by a chicken embryo DNA virus on the chorio-allantoic membrane compared to controls (Singh, L. M. and Gupta, G. 1985 Brit. Homeopathy 74:168-174). Other homeopathic medicines, the same medicines at different homeopathic potencies, or control phosphate buffered solution (PBS), had lesser or no effect.

While the exact mechanism of action of homeopathic medicines is unknown, magnetic resonance image measurements on serial dilutions of substances indicate that the hydroxyl (OH) groups in the solvent of solutions continue to change as dilutions become successively higher (Sacks, A. D. 1983 J. Holistic Med. 5:175-176; Smith, R. and Boericke, G. 1968 J. Am. Inst. Homeopathy 61:197-212; Smith, R. and Boericke, G. 1966 J. Am. Inst. Homeopathy 59:263-279). It is clear that the specific effects of homeopathics are of a non-molecular origin, yet provide potent biological activities that are clinically effective. It has been postulated that highly dilute compounds transfer biological activity to cells by electromagnetic fields (Benveniste, J. 1993 Frontier Perspectives 3:13-15). Del Giudice et al. have hypothesized that interactions between the electric dipoles of water and the radiation fields of a charged molecule generate a permanent polarization of water which becomes coherent and has the ability to transmit specific information to cell receptors, somewhat like a laser (Del Giudice, E., Preparata, G., Vitiello, G. 1988, Phys. Rev. Lett. 61:1085-1088).

Certain hormones have been prepared and used homeopathically. Adrenalinum, or ephinephrine, a sympathomimetic hormone produced by the medulla of the adrenal glands, thyroidinum, a preparation from the thyroid gland, and adrenocorticotrophin, or cortocotropin, a polypeptide hormone that increases the rate of secretion of the adrenal corticosteroids, are included in the official Homeopathic Monographs from the General Pharmacy of the Homeopathic Pharmacoepia of the United States. Insulin, an active molecule found in the pancreas which affects sugar metabolism, is listed in Boericke's Materia Medica, and is noted for its applicability for skin conditions. Parathyroid hormone, an extract from the parathyroid gland; thyreotrophic hormone, an extract from the anterior lobe of the pituitary gland; Corticotrophin, also extracted from the anterior lobe of the pituitary gland; cortisone and corticoids, which are steroid hormones; and folliculinum, a hormone secreted by the ovaries, are listed in the Materia Medica of New Homeopathic Remedies by Julian. The clinical symptomatology for parathyroid hormone includes general weakness, depression, asthenia, hypotonia, fatigue, pallor and emaciation. The clinical symptomatology for thyreotrophic hormone includes various conditions of the mind, digestive system, circulatory system, respiratory system, sense organs, and urinary and genital organs. The clinical symptomatology for corticotrophin includes various psychological and nervous conditions. The symptomatology of cortisone and corticoids includes various psychological, nervous, endocrine and digestive system conditions. The clinical symptomatology for folliculinum includes various conditions of the mind, digestive system and circulatory system.

### SUMMARY OF THE INVENTION

The applicant has previously demonstrated the clinical safety and efficacy of homeopathic preparations of various purified growth factors, including growth hormone. During the course of this work, and in efforts to identify homeopathic preparations that are yet more effective for various clinical conditions, the applicant has discovered that various other types of proteins and peptides (these terms are used interchangeably herein) may be formulated as homeopathic preparations and are highly efficacious in treating various conditions. The proteins are preferably highly purified and may be delivered, in a homeopathic preparation, via any suitable carrier or delivery system, including orally, topically, transdermally, via the lungs or nasal passages, via injection, or the like. Multiple proteins may be formulated in combination, in one or more homeopathic potencies. Homeopathic preparations of the present invention are non-toxic and do not produce undesirable side effects. They can be formulated and provided to a large patient population at a reasonable cost by means of delivery systems that are convenient and safe. Suitable proteins comprise a diverse group but, in general, are active in cell signaling, cell growth, development, differentiation and/or death, and regulatory functions.

In one aspect, homeopathic preparations of the present invention comprise a homeopathic potency of purified growth hormone. Growth hormone, or somatotropin, is a well-characterized single chain alpha-helical polypeptide containing a discrete receptor-binding domain. Human growth hormone is the most abundant hormone secreted by the anterior pituitary gland and has significant anabolic and anti-catabolic effects on the body. Cells of the immune system, such as macrophages and lymphocytes also produce and secrete growth hormone. Growth hormone stimulates the liver to produce somatomedins which, in turn, promote bone, muscle, cartilage, kidney, liver and skin growth. Two cleaved forms of human growth hormone have been isolated, one has prolactin-like activity and the other has growth promoting activity greater than that of the uncleaved molecule.

Additionally, homeopathic formulations of purified growth hormone are effective in reducing blood pressure and improving cardiovascular function; increasing serum IGF-1 is levels; treating growth deficiency disorders; increasing lean body mass, muscle mass and physical strength; improving pulmonary function, vascular and intracellular nutrient support; revitalizing liver, spleen, and brain functions; increasing libido and sex hormones; improving lipoprotein balance and fatty acid levels; increasing energy levels, oxygen uptake, nitrogen retention, physical mobility and exercise performance; reducing joint, back and knee pain; reducing joint swelling; eliminating cellulite and improving cholesterol profile; promoting hair growth and color change; reducing bleeding of the gums, nasal and sinus congestion; improving dermal cellularity and collagenicity; increasing cartilage strength; increasing the size an function of the thymus and spleen; enhancing immune system function and lymphocyte count; and reducing fat, in particular, hip and waist size. Homeopathic preparations of the present invention furthermore may be used to improve short term memory; reduce the manifestations of anger, anxiety, depression, social isolation, mood swings and sleeping disorders, improve vision, remove wrinkles, quicken wound healing, breast enlargement, and generally contribute to a feeling of well-being. Additionally, homeopathic preparations of the present invention may be used to treat headaches, AIDS wasting syndrome, Turner syndrome, osteoporosis, Parkinson's, Alzheimer's disease, Down's syndrome and autism.

Other purified proteins that may be used alone in a homeopathic formulation, or in combination with purified growth hormone in a homeopathic formulation, include: growth factors described in prior related patents that are incorporated herein by reference, particularly insulin-like growth factors such as insulin-like growth factor-1 (IGF-1), otherwise known as somatomedin and sulfation factor and related proteins; Fibrinogen β; glycoprotein 130 (GP130); signal transducer and activator of transcription 3 (STAT3); mitogen activated protein kinase p38 (p38MAPK); growth arrest and DNA damage inducible protein 45 (GADD45); apurinic endonuclease (APEN); membrane-type 1 matrix metalloproteinase-transmembrane protein (MTI-MMP); monocarboxylate transporter 1 (MCT1); fatty acid binding protein (FABP); epidermal growth factor receptor (EGF-R and transforming growth factor-alpha receptor (TGF-alpha-R); insulin-like growth factor binding proteins 1 and 3 IGFBP-1 and IGFBP-3; acid labile subunit of the IGF binding complex (ALS); suppressors of cytokine signaling (SOCS); transcription factors c-fos, c-jun, interferon response factor (IRF)-1, and hepatocyte nuclear factor-6 (HNF-6). Combinations of homeopathic potencies of purified growth hormone with purified insulin-like growth factor-i are especially preferred for many applications.

In another aspect, homeopathic preparations of the present invention comprise a homeopathic potency of at least one of the following classes of growth factors: insulin-like growth factors. (IGF); platelet derived growth factors (PDGF); transforming growth factors (TGF); granulocyte macrophage-colony stimulating factors (GM-CSF); epidermal growth factors (EGF); and fibroblast growth factors (FGF). Preparations comprising a homeopathic potency of fibroblast growth factor-2 (FGF2) and fibroblast growth factor-1 (FGF1) are especially preferred. FGF2 is a member of a large family of proteins that bind heparin sulfate and modulate the function of a wide variety of cells especially in terms of growth and development, survival, stimulation of new blood vessels (neovascularization) and cell specialization (Nugent & Iozzo 2000; Okada-Ban et al. 2000). It used to be called basic FGF to distinguish it from the acidic FGF (FGF1). FGF2 has been identified as a regulator of G-proteins and is well known for its activation of tyrosine (a required rate-limiting precurser amino acid in the formation of serotonin and the synthesis of catecholarines) via adenylate cyclase signaling. There is a direct relationship between FGF2 activity and serotonin uptake activity. FGF2 is widely distributed in the brain, and in the rat (a model with similarities to humans) FGF2 is found at highest levels in astrocytes with FGF2 receptors on oligodendrocytes as well. The hippocampus, amydala, hypothalamus, mesencephalic trigeminal nucleus and cells of the cerebellum and the lateral walls of the III ventricle in the brain all have both FGF2 and FGF receptors. The nurse cells surrounding the neurons and the white matter have FGF2 and their receptors.

In addition to FGF2, other important growth factors participate in nervous system healing and protection. IGF-1 is produced throughout the brain, especially in the cerebral cortex, hippocampus, cerebellum and diencephalons (Lee et al. 1999; Ye & D'Ercole 1998). IGF-1 regulates and protects neuronal cell growth, healing and differentiation. It is particularly important during post-natal development even under conditions of under-nutrition. IGF-1 can ameliorate brain growth retardation caused by lack of nutrients or toxic agents. IGF-1 prevents cerebellar granule cells from developing neurotoxicity (Leski et al. 2000) and IGF-1 plays critical roles in neuronal survival by regulating which neurons live, die (via apoptosis) or specialize (differentiate). IGF-1 regulates neurite cell length (Raghunath et al. 2000). IGF-1 is also produced throughout the body with the highest levels produced in the liver (commonly, in response to human growth hormone). It is also produced in the intestines, kidney, spleen, pancreas, lung, heart, testes, and my macrophage immune cells.

As a widely effective hormone/growth factor/neuropeptide, IGF-1 generally exerts its effects on growth and healing, especially in the liver, muscles, intestines and in the nervous, immune, and hormonal systems. IGF-1 regulates which cells progress into DNA synthesis. IGF-1 exerts its regulatory and cell-signaling effects without the necessity of entering the cell through activation of specific, high affinity, cell-surface receptors. Homeopathic preparations of IGF-1, alone or in combination with other purified proteins described herein, are highly efficacious for treatment of various conditions and repair of DNA damage.

PDGF plays a critical role in the timing and differentiation (specialization) of multi-potent stem cells into astrocytes of oligodendrocytes especially during late fetal and post natal development. It plays an important role in regulating FGF activity and is found along the micro-blood vessels in the brain. Its expression is highest in neuronal cell bodies of the cerebellum, cerebral cortex and hippocampus. PDGF stimulates nerve regeneration and glial cell proliferation. PDGF is called the competence factor because it moves cells out of a “resting phase” and activates them to enter the cell cycle. PDGF and IGF-1 work often together to move cells through the entire cell cycle to promote healing, regulate gene expression and maintain optimal homeostasis within the body. Homeopathic preparations of PDGF, alone or in combination with other purified proteins described herein, are highly efficacious for treatment of various conditions.

TGF_{β}1 plays a key on/off regulatory role throughout the body turning on and off other growth factor's effects depending upon the context of the environment surrounding specific cells. TGF_{β}1 plays a critical role with inflammatory processes, especially with respect to control of genetic regulatory sites associated with gene transcription. TGF_{β}1 is well known for its participation with wound healing by regulating an “off” signal to prevent overgrowth of cells. TGF_{β}1 is expressed within the cerebellum, hippocampus, hypothalamus and midbrain (Gayle et al. 1999). Unlike EGF, IFG-1 and PDGF, which drive cell cycle events by means of positive feedback, TGF_{β}1 generally regulates cell cycle events by means of a negative feedback mechanism. At the molecular level, there is good evidence that TGF_{β}1 induces both transcriptional and translational regulation of growth factors, such as TNF-α TGF_{β}1 is also important in regulating polyamines. Homeopathic preparations of transforming growth factors, such as TGF_{β}1, alone or in combination with other purified proteins described herein, are highly efficacious for treatment of various conditions.

According to yet another embodiment of the present invention, homeopathic preparations of the present invention comprise one or more homeopathic potencies of a purified nerve growth factor (NGF). Such preparations are suitable for treating illnesses such as chronically injured sensory afferent nerves in adult spinal cord, olfactory defects and sensory regression, microglial deactivation, HIV-associated sensory neuropathy, nerve damage, astrocyte or oligodendrocyte damage, dysfunction of cerebrospinal fluid volume and biological activity of children or adults, estrogen defects related to neurite outgrowth and cytoskeletal gene statement, damage to pancreatic beta cells, retinal photoreceptor and glial cells and visual damage, brain neocortex and striatum damage, hippocampal stem cell damage, latent or active HSV-1 damage to primary sensory neurons and apoptotic processes related to NGF receptor activity. NGF is crucial for cell survival.

According to still another embodiment of the present invention, homeopathic preparations of the present invention comprise one or more homeopathic potencies of a purified cyclin, such as an A or A-type cyclin, a B or B-type cyclin, a C or C-type cyclin, a D or D-type cyclin, or an E or E-type cyclin, alone or in combination with other proteins described herein. Preparations comprising one or more homeopathic potencies of one or more of the specified growth factors, or of one or more of the specified cyclins may be provided, as may preparations comprising one or more homeopathic potencies of one or more of the specified cyclins in combination with one or more of the purified growth factors specified. Cyclins are suitable for supporting retinoic acid-mediated growth, statement of CD26, diseases related to cell cycle arrest at various phases of the cell cycle, apoptosis, p53-dependent transcription in tumor cells, retinoblastoma gene protein (pRB) statement, and metastasis during carcinoma. Cyclins also play key roles in cell cycle control during such disease processes of obsessive compulsive disorder, autistic spectral disorder and Down's syndrome.

In particular, combinations of one or more proteins described above with one or more growth factors are preferred for particular applications, the growth factors including granulocyte macrophage-colony stimulating factors (GM-CSF), granulocyte-colony stimulating factors (G-CSF), macrophage-colony stimulating factors (M-CSF), tumor necrosis factors, hepatocyte growth factors, insulin-like growth factors (IGF), transforming growth factors (such as TGF-_{β}), nerve growth factors (NGF), epidermal growth factors (EGF), stem cell factors (SCF), platelet-derived growth factors (PDGF), fibroblast growth factors (FGF), interleukin-1, interleukin-2, keratinocyte growth factors, ciliary neurotrophic growth factors, Schwann cell-derived growth factors, vaccinia virus growth factors, bombyxin, neu differentiation factor, v-Sis, glial growth factor/acetylcholine receptor-inducing activity and other proteins belonging to their structural superfamilies.

Fragments or epitopes of the proteins described above may be used in place of the full-length proteins. Analyses of human growth hormone, for example, have characterized important functional epitopes for binding to its native receptor, human growth hormone binding protein (hGHbp). Approximately 31 side chains of hGH participate in the receptor-ligand interaction, but only eight of these side chains account for 85% of binding energy (Fregly, M. J., and Luttge, W. G., 1982, Human Endocrinology: An Interactive Text, Elsevier Biomedical, New York). According to another aspect, functional, or biologically active, fragments and/or epitopes of the proteins described above are used in homeopathic preparations of the present invention.

In one aspect, homeopathic preparations of the present invention may be suitable for treating disorders caused by human immunodeficiency virus (HIV). HIV competes for the same G-protein signaling processes that many growth factors use. It is known that G-proteins are requisite co-receptors for HIV in order that human cells can become infected (Feng et al. 1996; Alkhatib et al 1996; Balter 1996). G-proteins are the first site of information transfer from the cell membrane surface to its DNA (Conklin and Bourne 1993). Information from outside of cells to their inner nucleus use common pathways such as G-proteins. HIV, electromagnetic forces, and homeopathic growth factors all use G-proteins to transfer information to the DNA. Cells process a broad array of diverse extracellular signals into an appropriate small array of intracellular responses through only three types of signaling pathways: receptors, G-proteins, and effectors that direct the information from the receptor into the cell (Linder and Gilman 1992, Birnbaumer 1992; Restrepo et al. 1990). G-proteins are part of a superfamily of proteins, thus named by their binding and regulation by guanine nucleotides (which consist of a guanine, a sugar and one or more phosphates). There are three different subunits to G-proteins (Rahmatullah et al. 1995) which form a variety of combinations to direct various signals down specific pathways. There are over 320 heterotrimeric G-proteins (Birbaumer 1992, Conklin and Boume 1993). The signal transduction pathway of G-proteins is known to associate with numerous diseases (Milligan and Wakelam 1992, Karin 1992). HIV may activate G-proteins in the same way as radio frequency signals and homeopathic growth factors do. In fact, this signal transduction pathway may be one of the common pathways used universally.

G-proteins play an essential role during HIV infection. Researchers have known since 1986 that the CD4+ T lymphocyte receptor was not sufficient for HIV entry into immune system cells of humans (Balter 1996). This finding launched a decade of research into the necessary ‘second receptor’ for effective HIV infection into CD4+ T-cells of the immune and nervous systems. Researchers have recently identified at least two ‘second receptors’ that enable HIV infection in human cells; both of them are transmembrane G-proteins that couple with the CD4+ receptor, called CC-CKR5 and fusin (Feng et al. 1996, Alkhatib et al. 1996). Now, CC-CKR5 and fusin are accepted as the second and final gate that HIV must pass through for entry into the macrophage and CD4+ cells, infecting them by insertion of viral genetic material into a cell's DNA.

G-proteins take part in an enormous variety of biological sensing and communication systems. They help control everything from successful entry of HIV into human immune cells, to mating in yeast, to egg-laying in the nematode Caenorhabditis elegans, to immune function, vision, and olfaction in humans (Roush 1996). G-proteins are located within the cell lipid bilayer and are part of a superfamily of proteins (Gilman 1987). G-proteins couple with cell receptors to direct the flow of information from outside the receptor to the inside of the cell by converting the external signal into second messengers that regulate gene expression (Linder and Gilman 1992, Karin 1992). G-protein activation triggers signal transduction cascades, which regulate gene transcription, gene expression, protein synthesis, cell division, and cell death (Rubik et al. 1994, Goodman et al. 1994, Cadossi et al. 1988, Cone 1970). G proteins play significant roles during infection by viruses such as HIV, Epstein Barr virus, and bacterial diseases such as some lymphomas, whooping cough, and cholera. Other G-protein-coupled-receptor disorders are well known, such as retinitis pigmentosa, stationary night blindness, color blindness, isolated glucocorticoid deficiencies, hyperfunctioning thyroidadenomas, and neonatal hyperparathyroidism. G-proteins contribute in significant ways to receptor systems as broad as growth factors, growth hormone, chemokines, neurotransmitters, hormones, and gonadotropin-releasing hormone, phermones, opsins and rhodopsins, melatonin and serotonin.

Since electromagnetic forces are necessary co-factors for successful HIV infection, a treatment strategy for HIV may include competitive G-protein signaling to inhibit HIV replication and activation. These types of approaches utilize homeopathic cell signaling proteins, such as homeopathic growth factors and homeopathic interleukins.

In another aspect, homeopathic preparations of the present invention are especially suitable for treating various neurological disorders, such as autism and Down's syndrome. Autism is a neurobiological disorder whereby autistic individuals do not communicate or respond in the same manner as the general population. The incidence of autism is four to five times higher in boys than in girls. Autistic individuals have developmental delays that impair social interactions, impair verbal and non-verbal communications, such as lack of eye contact, speech difficulties and openness for social interactions. The individuals also enter into repetitive and stereotypical patterns of behavior and appear to have no fear of societal definitions of danger. The ability to identify self from non-self is low. It appears as if these individuals have low tolerance for frustration, poor comprehension of communications toward them, exhibit poor skin color, reveal little awareness of their surroundings and have a low interest level in their interactions with the world outside themselves. The sleep disturbance that is most common is early morning arousal (Hering et al. 1999).

The cause of autism is unknown and thus is open to many different types of theories ranging from environment toxicity, to viral infections and biochemical to developmental imbalances. Seizures and other co-existing nervous system and digestive system imbalances are common. Some individuals with autism do respond to subtle energy interventions and communications, such as hands-on-healing (Reiki), cranio-sacral therapies, and biofeedback. Some individuals respond to nutritional interventions and behavioral educations. It is entirely probable that autism is not caused by a single agent or can be profiled in the same way, thus the same treatments will not work for every individual or to the same degree. It is clear that autism includes damage to the DNA and genes.

Studies on toxic environmental chemicals show a statistical significance in children and their families (Edelson & Cantor, 1998; Felicitti, 1981; Niewander and Gordon, 1972). Drs. Edelson and Cantor examined 20 autistic children and showed that all children exhibited chronic toxicological damage, especially in the intestines, liver and tissues of the central nervous system.

Brain research is scarce and has enough inconsistencies to prevent a universal conclusion as to the site(s) or causes of autism. However, it is believed that anatomical defects in autism are caused by abnormal development in areas of the brain versus damage to fully developed brains. The areas of the brain that are affected include the cerebellum, the hippocampus and the frontal and temporal lobes of the cerebral cortex, especially those areas related to memory and emotional systems (the limbic system). The abnormalities found include the stunting of dendrites; abnormal secondary and tertiary branching of dendrites and reduced numbers of Purkinje cells (Arin et al. 1991; Bauman & Kemper 1985).

At the biochemical level of understanding the autistic brain, it appears to be generally agreed upon that serotonin synthesis is depressed in the frontal cortex and the thalamus, while serotonin is elevated in the dentate nucleus of the cerebellum (Buitelaar & Willemsen-Swinkels, 2000; Rumsy & Ernst, 2000). The neurotransmitters related to dopamine also are implicated as out of balance, especially in the frontal lobes of the cerebrum and the cerebellum (Rumsy & Ernst, 2000). In general, damage in the nervous system includes that to dendrites, neurons, axons, myelin and oligodendrocytes. The cerebral cortex controls higher cognitive functions. Connections between the cortex and the basal ganglia control the motor and cognitive programs, whereas connections between the cortex, the amydala and medial temporal lobes mediate emotional behavior.

There are some theories that autoimmune processes have played a role in the ongoing problems of autism. One study demonstrated that CD4 lymphocytes and their naïve recruit lymphocytes (CD4+ CD45RA+) are very low in autistic individuals. Natural killer cells are also decreased in autism (Kalf et al., 1982; Pangborn, 1984; Warren et al., 1985; Yonk et al., 1990). Thus, transcriptional or translational control is lacking or dysregulated.

The body is challenged daily by a barrage of toxins and changing pathogens. Our sense of well being and survival are maintained in tact via a highly regulated cell-to-cell communication network within the neuro-immuino-endocrine system. This system uses the language of growth factors (also known as cytokines) to coordinate activities of the immune, nervous and hormonal systems. The neuroimmunoendocrine system is adaptive and memory-specific to each person's set of experiences. Development and maturation occur as the regulatory controls over cell-to-cell communication strengthen. Premature aging occurs once well-established regulatory controls over cell-to-cell communication break down. It is possible to strengthen the regulatory controls over cell signaling with the brain, immune and hormonal (endocrine) systems to improve health and quality of life, and to build a sense of self in relationship to the surrounding world using the homeopathic growth factor preparations of the present invention.

According to one embodiment of the present invention, one or more homeopathic preparation(s) are administered for the treatment of a neurological disorder, such as autism. A homeopathic preparation comprising a fibroblast growth factor, preferably FGF-2, may be administered in combination with a homeopathic preparation comprising IGF-1, PDGF_{ββ} and TGF_{β}1, for example. Alternatively, a homeopathic preparation comprising one or more homeopathic potency of each of the following constituents may be administered: a fibroblast growth factor such as FGF-2; IGF-1, PDGF_{ββ} and TGF_{β}1. In alternative embodiments, multiple fibroblast growth factors may be combined with one another and/or one or more of the specified growth factors.

Numerous oral delivery formats are known. Various diluents may be used to prepare liquid forms of the inventive homeopathic preparations, depending on the desired delivery system. In one embodiment, liquid forms of the inventive homeopathic preparations may be in a form of a solution of purified water, glycerin, citric acid, and a preservative such as sodium benzoate, a solution of purified water, glycerin, potassium sorbate, and/or a form of proteinated-copper in a cationic state, and a preservative such as sodium benzoate, and the like. Other diluents for oral delivery, including various alcohol-containing solutions, are known in the art and may be employed in the present invention to increase solubility and stability of purified growth hormone.

In another embodiment, solid forms of the inventive homeopathic preparations are prepared in pellet or tablet forms. The pellets and tablets are made from a suitable organic material, such as lactose or sucrose by methods well known in homeopathy. In particular, tablets are generally produced in two forms, as tablet triturates or compressed tablets. The tablets or pellet forms of the homeopathic preparations are then crushed, grinded, or granulated and introduced into and combined with the manufactured food products as described above.

In another embodiment, various potencies and combinations of homeopathic preparations of the purified growth hormone and purified growth factors described above are delivered in an oral delivery format in combination or association with a liquid or solid carrier. In a preferred format, homeopathic preparations of purified growth factors, growth hormone, cyclins and other substances described herein are formulated in association with food or beverage carriers to provide a food or beverage item comprising a homeopathic preparation. Liquid forms of the homeopathic preparations may be introduced into and combined with foods and beverages to provide the homeopathic constituents of food and beverage products of the present invention. Alternatively, solid forms of homeopathic preparations such as tablets may be ground and combined with foods and beverages to provide the homeopathic constituents of food and beverage products of the present invention.

Generally solid food carriers may include, and are not limited to, fruits and fruit products, dairy and dairy products, and food products such as chocolate, candy bars, energy bars, protein snacks, other types of food products, and the like. Chocolates of various types are a preferred solid food carrier associated with homeopathic preparations of the present invention. Generally liquid beverage carriers may include sports drinks, nutritional drinks, supplemented waters, fruit drinks, teas, chai drinks, and other liquids. Generally solid and liquid food and beverage carriers comprising one or more homeopathic potency(ies) of one or more growth factor, growth hormone, cyclin or the like, may additionally comprise one or more additional supplemental constituents, such as vitamins, minerals, amino acids, traditional homeopathic compositions, and the like.

In another application, homeopathic preparations of purified growth hormone formulated in a topical gel formulation were demonstrated to improve skin tone and skin conditions, including reducing the incidence of oily and sagging skin, reducing wrinkles, and increasing the resiliency and attractiveness of skin. Homeopathic preparations of purified growth hormone are useful for cosmetic applications, for treatment of burns and other skin conditions, for use as an anti-ageing agent, for treatment of skin conditions produced or exacerbated by environmental toxins and other environmental factors, including radiation, and generally to improve skin condition and appearance. Homeopathic preparations of purified retionic acid are also suitable for use in topical formulations, alone or in combination with purified growth hormone. Homeopathic preparations of α-glycerophosphorylcholine (α-gpc) are also suitable for use in topical applications, alone or in combination with purified growth hormone.

### DETAILED DESCRIPTION OF THE INVENTION

Homeopathic preparations of the present invention are expressed herein in homeopathic potencies, which are well recognized in the art of homeopathy. The relationships between homeopathic potencies and molar concentrations are not linear, since the potency of the starting material may vary in the preparation of homeopathic formulations. General statements regarding molar concentrations of proteins in the homeopathic preparations of the present invention may, however, be made. Thus, homeopathic preparations of the present invention preferably comprise a concentration of 1×10⁻⁶ M purified protein or less and, alternatively or additionally, comprise a concentration of 1×10⁻¹² M purified protein or less, preferably a concentration of 1×10⁻²⁴ M purified protein or less, and most preferably comprise a concentration of 1×10⁻⁶⁰ M purified protein or less. Homeopathic preparations of the present invention preferably comprise a homeopathic potency of purified growth hormone of one or more of the following potencies: 6 X; 6 C; 7 C; 5 X; 12 C, 30 C, 100 C, 200 C and 1M. Other homeopathic potencies are known in the art of homeopathy and may be used in preparations of the present invention. Homeopathic preparations of the present invention are defined as comprising a purified protein if the preparation is derived or originated from a preparation comprising a measurable quantity or activity of a purified protein, preferably, a recombinantly-produced and highly purified human protein having a specified bioactivity based on World Health Organization (WHO) reference standards.

Purified protein(s) for use in homeopathic preparations of the present invention may be isolated from natural sources, or may be produced using recombinant techniques or other protein synthesis technology. Proteins isolated from mammalian sources, or produced recombinantly and having substantially the same structure and activity as human proteins, are preferred. Recombinantly produced proteins are especially preferred. The purity of the protein(s) used in homeopathic preparations of the present invention is preferably at least about 90%, and more preferably at least 95%, and most preferably at least 98%. Human proteins are preferred, although proteins that are different from but have a high degree of similarity to human protein molecules are suitable. Purified recombinant proteins are available commercially. Purified recombinant human growth hormone, for example, is commercially available from several sources, including NovoNordisk Eli Lilly, Pharmacia/Upjohn, Ares Serono and Genentech. In addition to overall purity, the biological specific activity of purified proteins is important, based on World Health Organization (WHO) reference standards.

Homeopathic preparations according to the present invention may contain multiple potencies of an individual purified protein, or multiple purified proteins, or multiple potencies of multiple proteins, and/or other purified constituents, such as vitamins, supplements, etc., in addition to the purified protein. Especially preferred homeopathic preparations of the present invention comprise multiple potencies of purified protein(s), including 6 X, 7 C, and 12 C potencies, as well as a combination of 6 C with 100 C and 200 C potencies, and a combination of 6 C with 1M potencies. In the Example provided below, a homeopathic preparation of purified recombinant human growth hormone was formulated in a gel formulation at multiple homeopathic potencies of 8 X+15 X+24 X(12 C)+6 C.

Various potencies of one or more purified protein(s) may be combined with other purified constituents, such as vitamins, minerals, amino acids, and/or traditional homeopathics, the purified constituents having a potency greater than or equal to 3 X. Substances including carnitine, CoQ10, vitamin K, vitamin E, Ginko Biloba, Phosphadityl Serine, Omega 3 oils, flax seed, fish oil, polyamines, alpha hydroxy, glycolytic acid, and DHA are preferred and optimal constituents of the homeopathic growth factor preparations of the present invention. Examples of traditional homeopathics that may be used in combination with homeopathic preparations of the present invention include arsenicum, pulseatilla, aconite, hypericum and metabolic sarcodes. The same potencies, or combinations of potencies, of homeopathic preparations of purified protein(s) of the present invention and additional constituents are used regardless of whether the administration is in liquid, solid, spray, topical, transdermal or injectable form. Additional components, such as stabilizers, buffers, preservative compounds, etc. may be used and are well known in the art, as described in the United States Homeopathic Pharmacopoeia.

Homeopathic preparations comprising a purified protein may be administered orally, in liquid, gel or cream, or solid form, such as pellets or tablets or in association with generally solid food or generally liquid beverage carriers. Oral administration is convenient and effective. Alternative delivery systems, such as eye drops, nasal sprays, throat sprays, topical preparations, transdermal delivery, and injectable (intracutaneous, intramuscular, intravenous, or subcutaneous) delivery systems also provide convenient and effective delivery of the homeopathic preparations of the present invention.

Oral delivery of polypeptides and proteins is generally ineffective, since the polypeptides and proteins are generally broken down and rendered inactive in the blood stream before they reach their desired target or exert their desired effect. Traditionally, polypeptides and proteins are thought to be effective only when delivered via injection, intranasally or intravenously, and undesirably high dosages are administered because a large proportion of the delivered dosage is destroyed prior to exerting its effect or reaching its target. The mechanism of action of the homeopathic preparations of the present invention comprising a purified protein has not been fully delineated, but it is generally thought to exert its effects through a non-molecular mechanism using signal transduction pathways carrying signals into the cell's nucleus where the cell cycle and DNA transcriptional and translational controls are affected. It is postulated that homeopathic preparations transfer biological activity to cells by electromagnetic fields. Although the mechanism is undefined, the clinical effects of such preparations, delivered orally using liquid or chewable tablet formulations, have been demonstrated in numerous patients, and demonstrated in double-blind, placebo controlled studies described in prior related patents that are incorporated herein by reference.

Various diluents or substrates may be used, depending on the desired delivery system. Appropriate diluents for the following delivery systems are well known: oral administration in liquid or solid form; eye drops; nasal sprays; throat sprays; injectables; topical preparations; cosmetic preparations; and transdermal preparations. Homeopathic potencies of purified protein(s) may be provided in products that are used on a daily basis, such as tooth pastes and gels, nail creams or gels, ear drops, cosmetics, head and body shampoos and conditioners, vaginal and anal suppositories, and the like. One or more potencies of purified protein(s) and/or one or more additional constituents, such as vitamins, minerals, amino acids, or traditional homeopathic preparations, may be combined in a preparation.

Preferred diluents for “standard” oral administration of homeopathic preparations of the present invention include solutions of purified water, glycerin, citric acid and a preservative such as sodium benzoate; or a solution of purified water, glycerin, potassium sorbate, and/or a form of proteinated-copper in a cationic state, and a preservative such as sodium benzoate. Other diluents for oral delivery, including various alcohol-containing solutions, are known in the art and may be employed in the present invention to increase solubility and stability of purified growth hormone.

In another embodiment, homeopathic preparations of the present invention for oral administration are formulated in association with a generally liquid beverage carrier such as a water drink, fruit juice, sports drink, nutritional drink, milk or dairy product, tea, chai, carbonated beverage, coffee drink, liquor or the like. And, in yet another embodiment, homeopathic preparations of the present invention for oral administration are formulated in association with a generally solid, food-related carrier such as chocolate, candy or energy bars, protein snacks, dairy products and other food-based carriers.

The homeopathic preparations of the present invention are preferably administered orally but may also be prepared in topical formulations for application to the skin; administered transdermally; by intracutaneous, intramuscular, intravenous, or subcutaneous injection; or administered in the form of eye drops or nasal and throat sprays. Lotions for topical and transdermal application, and buffered salt solutions for eye applications, are well characterized and widely used in the cosmetic industry, which are readily adaptable to the preparation of the present invention. Additionally, carrier solutions for intranasal administration of substances are well known in the art and widely used in drug delivery systems.

In one embodiment, homeopathic preparations of purified protein(s) are prepared in a chewable tablet form. The tablets are made from a suitable organic material, such as lactose or sucrose (Dolisos, Las Vegas, Nev.), by methods well known in homeopathy, as described in the United States Homeopathic Pharmacopoeia. In particular, tablets are generally produced in two forms, as tablet triturates or compressed tablets. Tablet triturates are produced by preparing a homeopathic preparation of purified growth hormone, as previously described, and adding binders as necessary. Binding solutions are composed of a binder, such as gum arabic, microcrystalline cellulose, a preservative if necessary, an inert lubricant, and purified water. The tablets are then molded by hand or preferably by automated equipment, and the tablets are then dried by introducing them into a dehumidified environment with a relative humidity of 35-40%, and an ambient temperature of 70 to 110° F. Compressed tablets are formed by compression of a dry material and contain no special coating. They are compressed from powdered or crystalline solids, and, as with tablet triturates, may contain binders, excipients, lubricants, and disintegrators. Compressed tablets are produced by adding the homeopathic preparation of purified growth hormone to the lactose preparation until thoroughly moistened. Binders may be added at this time as necessary, as described above for tablet triturates. The moistened material is granulated by passing through an appropriate mesh screen, and the moistened granulation is introduced into a dehumidified environment and subsequently dried as described above. The dried granulation is then regranulated through the mesh screen and lubricants, such as mineral oil, talc, calcium stearate, corn starch, are added as necessary. The mixture is then compressed in a rotary tablet compressor or any similar apparatus to the desired tablet size.

In one embodiment, homeopathic preparations of the present invention comprise a generally solid food-based or generally liquid beverage-based carrier in combination with a homeopathic potency of one or more compositions selected from the group consisting of: insulin-like growth factors including IGF-1, growth hormone, PDGF_{ββ}, GM-CSF, M-CSF, transforming growth factors including TGF_{β}1, fibroblast growth factors including FGF1 and FGF2, nerve growth factors (NGF) and epidermal growth factors such as EGF. One or more of the following homeopathic potencies of the one or more compositions may be associated with the generally solid food-based or generally liquid beverage-based carriers: 6 X, 6 C, 7 C, 5 X, 12 C, 30 C, 100 C, 200 C and 1M, and combinations thereof. Homeopathic potencies of 6 C and 7 C are preferred for some generally solid food-based carriers such as chocolates.

In other embodiments, food-based generally solid carriers and beverage-based generally liquid carriers may be associated with one or more of the following specific homeopathic preparations: PDGF_{ββ} (30 C+1M potency); TGF_{β}1 (30 C+1M potency); GM-CSF (200 C potency); recombinant hGH (6 C+100 C+200 C potency); recombinant hGH (6 X +12 C potency); recombinant EGF (12 C+30 C potency); 30 C+1M potency of a combination of recombinant IGF-1, PDGF_{ββ}, TGF_{β}1; FGF-2 (6 C+1M potency); recombinant human fibroblast growth factor 1 (FGF-1); and a combination of recombinant human IGF-1 (30 C+1M potency), PDGF_{ββ (}30 C+1M potency), TGF_{β}1 (30 C+1M potency), and GM-CSF (200 C potency).

In another embodiment, homeopathic preparations of the present invention are formulated on a topical delivery system, such as a lotion, cream, gel (including gels for use in proximity to the eye or nose), droplets (including droplets in a nasal spray), cosmetics, and the like. These formulations are particularly useful for treatment and improvement of skin conditions. The results of various clinical trials demonstrating the efficacy of various homeopathic preparations of purified protein(s) are presented in the patents incorporated herein by reference. The following example represents the results of additional clinical: studies and supports additional therapeutic uses of preparations of the present invention.

### EXAMPLE 1

We evaluated the unique cosmetic effects of topical application of homeopathic recombinant hGH (HrhGH) formulated in an established botanical cosmetic eye gel compared to effects of botanical eye gel alone and compared to a placebo eye gel. Evaluation was based upon 19 self-observable characteristics of the skin and peri-orbital skin around the eyes with respect to improved under eye skin appearance, moisture and beauty. Cellular drug effects were also evaluated using 17 self-observable symptoms. HrhGH source material was obtained as purified recombinant human growth hormone from Novo Nordisk, Gentofte, Denmark, and was prepared homeopathically with hand successions to potencies of 8 X+15 X+24 X+6 C in a water base, which was mixed with a botanical gel formulation.

Three groups of participants were randomly placed into each of the three arms of the study: placebo, eye gel alone or eye gel+ HrhGH. Eye gel was applied topically in the morning under the eyes. Participants scored each of 40 characteristics from 0-10 on a weekly basis, including baseline, for six weeks. Five statistically significant findings were made: (1) topical application of HrhGH decreased wrinkles; (2) topical application of HrhGH decreased sagging skin under the eyes; (3) topical application of HrhGH decreased oily skin; (4) topical application of HrhGH increased attractiveness of eyes; and (5) topical application of HrhGH and increased skin resilience under the eyes over time compared to the placebo group. There was no statistically significant finding regarding the effects of the botanical eye gel alone compared to placebo.

These results demonstrate unique topical benefits of nanograms and lower concentrations of HrhGH in an eye gel compared to injectable rhGH. HrhGH fulfills the Law pf Similars by decreasing oily skin which is a key characteristic of acromegaly, a disease of excess hGH. These findings, like others in homeopathy, suggest that a different mechanism of action may be operating than the conventional theory of receptor-ligand binding pharmacology. It is hypothesized that HrhGH at nanomolar concentrations may work via its unique biochemical electromagnetic properties on stratum corneum elastic fibers as well as at the level of the DNA. Like retinoic acid, HrhGH may work at the ultra-structural level to improve linearity of elastin fibers.

### EXAMPLE 2

Chocolate products incorporating a homeopathic potency of IGF-1, or sulfation factor, were manufactured and tested for their taste and salutary effects. The reactions of testers were generally positive and several are described below.

After tester 1 consumed the inventive chocolate product incorporating a homeopathic potency of IGF-1, or sulfation factor, he felt clearing of his sinuses and sensed warming in areas around his forehead, top of his head, and the back of his head. After a few minutes, tester 1 felt a form of awareness around the muscles in his upper arm. After consumption of the inventive chocolate product, tester 2 was aware of a sensation of warming about an area of his heart. The warming sensation traveled to an area about his forehead and his face, and tester 2 additionally felt a sense awareness and awakeness.

Tester 3, after consuming the inventive chocolate product, immediately felt gradual heating on his face and a sensation that traveled to the top of his head and the back of his head. Further, tester 3 felt calmness and an awareness of his heart.

Tester 4 consumed a 1.5 cm×1.5 sample of the inventive chocolate product and immediately felt a sensation and warming about areas around his forehead, top of the head, and back of his head. Tester 4 additionally felt a heightened sense of awareness and alertness.

The present invention has been described with reference to specific embodiments. These specific embodiments should not be construed as limitations on the scope of the invention, but merely as illustrations of exemplary embodiments. It is further understood that many modifications, additions, and substitutions may be made to the described homeopathic preparations and systems and methods of delivering such homeopathic preparations without departing from the broad scope of the present invention.

I Claim:

1. A preparation comprising a homeopathic potency of a purified composition selected from the group consisting of: insulin-like growth factor, insulin-like growth factor-1 (IGF-1), growth hormone, transforming growth factor (TGF), TGF_{β}1, fibroblast growth factor (FGF), FGF1, FGF2, GM-CSF, M-CSF, PDGF_{ββ}, nerve growth factor (NGF) and epidermal growth factor (EGF), wherein the concentration of said purified composition is less than 1×10-6 molar, and wherein said homeopathic potency is formulated in a generally solid, food-based or a generally liquid beverage carrier.

2. A preparation of claim 1 comprising a homeopathic potency of purified IGF-1.

3. A preparation of claim 1, wherein the homeopathic potency is formulated in a generally solid, food-based carrier selected from the group consisting of: a chocolate product, candy, an energy bar, a protein snack, and a dairy product.

4. A preparation of claim 1, comprising a homeopathic potency of purified IGF-1, wherein the food-based carrier is a chocolate product.

5. A preparation of claim 4 comprising a homeopathic potency of purified IGF-1 at a potency selected from the group consisting of: 6 X, 6 C, 7 C, 15 X, 12 C, 30 C, 100 C, 200C, 1M (1000 C), and combinations thereof.

6. A preparation of claim 4 comprising a homeopathic potency of purified IGF-1 at a potency selected from the group consisting of: 6 C and 7 C.

7. A preparation of claim 1 wherein the purified composition has a homeopathic potency selected from the group consisting of: 6 X, 6 C, 7 C, 15 X, 12 C, 30 C, 100 C, 200 C, 1M (1000 C), and combinations thereof.

8. A preparation as recited in claim 1 additionally comprising at least one component selected from the group consisting of: a vitamin, a mineral, an amino acid, carnitine, CoQ10, vitamin K, vitamin E, Ginko Biloba, Phosphadityl Serine, an Omega 3 oil, flax seed, fish oil, polyamines, alpha hydroxy, glycolytic acid, and DHA.

9. A preparation of claim 1, wherein the purified composition is at least 95% pure.

10. A preparation of claim 1, wherein the purified composition is a recombinant human protein.

11. A preparation of claim 1 comprising a combination potency formulation having at least two homeopathic potencies.

12. A preparation of claim 1, additionally comprising one or more traditional homeopathics selected from the group consisting of: arsenicum, pulseatilla, aconite, hypericum, and metabolic sarcodes.

13. A preparation of claim 1, comprising a homeopathic potency of purified growth hormone.

14. A preparation of claim 1, comprising a homeopathic potency of purified IGF-1, wherein the potency is 30 C.

15. A preparation of claim 1, comprising a homeopathic potency of purified IGF-1 and homeopathic potencies of purified PDGF and purified TGF_{β}1.

16. A preparation of claim 15, wherein the IGF-1, PDGF and TGF_{β}1 homeopathic potencies are formulated using human recombinant proteins.

17. A preparation of claim 1 comprising homeopathic potencies of purified IGF-1, purified GM-CSF, purified PDGF_{ββ} and purified TGF_{β}1.

18. A preparation of claim 17, wherein the IGF-1, GM-CSF, PDGF_{ββ} and TGF_{β}1 homeopathic potencies are formulated using purified, human recombinant proteins.

19. A preparation of claim 1, in a generally liquid beverage carrier selected from the group consisting of: a water drink, fruit juice, a sports drink, a nutritional drink, milk, a dairy product, tea, chai, a carbonated beverage, a coffee drink, a liquor, or combinations thereof.

20. A preparation comprising a homeopathic potency of purified insulin-like growth factor-1 (IGF-1) formulated in a chocolate carrier for oral delivery, wherein the potency of purified IGF-1 is selected from the group consisting of: 6 C and 7 C.
